(19) United States
(12) Patent Application Publication
(10) Pub. No.: US 2024/0076690 A1
(43) Pub. Date: Mar. 7, 2024

(54) VIRAL VECTORS AND USES THEREOF

(71) Applicant: SUNSHINE LAKE PHARMA CO., LTD., Dongguan, Guangdong (CN)

(72) Inventors: Xiaodan YANG, Dongguan (CN); Shiyou CHEN, Dongguan (CN); Xiuqin ZHU, Dongguan (CN); Junji DONG, Dongguan (CN); Xiaofeng CHEN, Dongguan (CN); Wenjia LI, Dongguan (CN)

(73) Assignee: SUNSHINE LAKE PHARMA CO., LTD., Dongguan, Guangdong (CN)

(21) Appl. No.: 18/028,029

(22) PCT Filed: Sep. 29, 2021
(86) PCT No.: PCT/CN2021/121509

§ 371 Date: Mar. 23, 2023

(30) Foreign Application Priority Data
Sep. 30, 2020 (CN) 202011064132.2

(51) Int. Cl. C12N 15/86; C07K 14/005; C07K 16/28; C12N 7/00

(52) U.S. Cl. CPC C12N 15/86; C07K 14/005; C07K 16/28; C12N 7/00; C07K 2319/30; C12N 2740/15043; C12N 2740/15052; C12N 2760/20222

(57) ABSTRACT

A group of viral vectors including: first viral vector, wherein first viral vector carries first nucleic acid molecule, and the first nucleic acid molecule encodes envelope protein; second viral vector, wherein second viral vector carries second nucleic acid molecule, the second nucleic acid molecule encodes fusion protein, the fusion protein includes single-chain antibody and C-terminal domain of envelope protein, the C-terminal domain of envelope protein includes transmembrane region and intracellular region of envelope protein, the C-terminus of single-chain antibody connects with N-terminus of C-terminal domain of envelope protein, the single-chain antibody targets a specific antigen; first nucleic acid molecule and second nucleic acid molecule are arranged to express envelope protein and fusion protein, and the envelope protein and the fusion protein are in non-fusion form. After the group of viral vectors are introduced into recipient cell, a virus with high viral titer can be packaged, and virus has targeted infectivity.

The present application contains a Sequence Listing that has been submitted electronically in ASCII format and is hereby incorporated by reference in its entirety. The ASCII copy, created on Jul. 13, 2023, is named Substitute Sequence Listing_ST25.txt and is 20,167 bytes in size.

### CROSS-REFERENCE TO RELATED APPLICATION

This application claims the priority and benefits of Chinese Patent Application No. 202011064132.2, filed with the State Intellectual Property Office of China on Sep. 30, 2020, which is incorporated herein by reference in its entirety.

### FIELD

The present invention relates to the field of biotechnology, specifically the present invention relates to viral vectors and uses thereof, and more specifically the present invention relates to viral vectors, lentivirus, pharmaceutical composition and method for introducing gene of interest into recipient cells.

### BACKGROUND

At present, as a gene delivery vector, lentivirus is widely used in gene therapy of various diseases, and is mainly concentrated in the field of ex vivo, such as CAR-T preparation, gene modification of hematopoietic stem cells, etc. Lentiviral vector cannot be widely used in the in vivo field, one of the main reasons is that the lentiviral transfection lacks targeting.

Researchers have been committed to achieving targeted transfection of lentivirus. From the published work, it is the envelope protein that determines the type of host cell transfected by the lentivirus. By changing the type or structure of the envelope protein, the lentivirus gains a tendency to transfect certain types of cells, but there are some shortcomings, In order to obtain the transfection tendency or targeting of the lentivirus, the researchers mainly make improvements in the following three aspects: first, the envelope protein is replaced with the envelope protein of other viruses with specific transfection tissues, so that lentivirus have acquired new transfection characteristics; however, the number of envelope proteins with clear functional background and guaranteed safety is limited, and the titer and stability of the obtained recombinant lentivirus have decreased. Second, the scFv domain of a receptor or antibody that specifically binds to an antigen is fused and expressed to the N-terminus of the envelope protein, or assembled into a viral vector as a separate membrane protein, so that the lentivirus can bind to the antigen through the receptor or scFv and has a tendency to transfect cells expressing the antige,; but this method greatly reduces the production titer of lentivirus (reduced by about 100 times). Third, the CD47 protein is co-expressed while packaging the virus to obtain a lentivirus (CD47hi LV) with a high expression of CD47 on the membrane surface. CD47 can interact with macrophage surface receptors to inhibit macrophages from phagocytosis of the lentivirus, thereby making CD47hi LV more efficient in gene transfer to hepatocytes and reducing the activation of acute inflammation response. However, this method uses the body's ability to metabolize the virus to passively increase the efficiency of CD47hi LV in gene transfer to hepatocytes, but it cannot actively target and infect hepatocytes.

In view of the fact that the current technology is still unable to solve the problem of the lack of targeting of lentivirus infection without significantly reducing the titer of the lentivirus, it is necessary to develop new technologies to solve this problem.

### SUMMARY OF THE INVENTION

The present invention aims to solve one of the technical problems in the related art at least to a certain extent. To this end, the present invention provides a viral vector with targeted infectivity and significantly improved virus titer.

In one aspect, provided herein is a group of viral vectors. According to the embodiments of the present invention, the group of viral vectors comprises: a first viral vector, the first viral vector carries a first nucleic acid molecule, and the first nucleic acid molecule encodes an envelope protein; at least one second viral vector, the second viral vector carries a second nucleic acid molecule, the second nucleic acid molecule encodes at least one fusion protein, the fusion protein includes at least one single-chain antibody and the C-terminal domain of the envelope protein, the C-terminal domain of the envelope protein includes a transmembrane region and a intracellular region of the envelope protein, the C-terminus of the at least one single-chain antibody connects with the N-terminus of the C-terminal domain of the envelope protein, the single-chain antibody targets a specific antigen; the first nucleic acid molecule and the second nucleic acid molecule are arranged to express the envelope protein and the fusion protein, and the envelope protein and the fusion protein are in a non-fusion form. According to the embodiments of the present invention, after the viral vectors are introduced into the recipient cells, a virus with high virus titer can be packaged, and the virus has targeted infectivity.

According to the embodiment of the present invention, the viral vectors may further comprise at least one of the following additional technical features:

According to the embodiments of the present invention, the viral vectors are retrovirus vectors, lentivirus vectors or other enveloped virus vectors.

According to the embodiments of the present invention, the enveloped virus comprises at least one selected from: Bornaviridae, Nyamaviridae, Arenaviridae, Filoviridae, Hantaviridae, Nairoviridae, Orthomyxoviridae, Paramyxoviridae, Bunyaviridae, Phenuiviridae, Rhabdoviridae, Arteriviridae, Coronaviridae, Flaviviridae, Togaviridae, Hepadnaviridae, Spumavirus, Iridoviridae, Herpesviridae, Poxviridae, and Deltavirus.

According to the embodiments of the present invention, the envelope protein is an envelope G glycoprotein (VW-GI) or a mutant of envelope G glycoprotein of vesicular stomatitis virus of the Rhabdoviridae. The envelope G glycoprotein of vesicular stomatitis virus has cell membrane adsorption and fusion capabilities. Therefore, the virus packaged by the viral vectors provided herein has cell adsorption and infection capabilities.

According to the embodiments of the present invention, the mutant of the envelope G glycoprotein has K47Q and R354Q mutations. The cell membrane adsorption capacity of envelope G glycoprotein mutant with K47Q and R354Q mutations is weakened, but its membrane fusion capacity is not affected. Therefore, the envelope G glycoprotein mutant with K47Q and R354Q mutations weakens the non-specific cell adsorption ability of the virus obtained by packaging, but it does not affect the ability of the virus to infects cells.

According to the embodiments of the present invention, the mutant of the envelope G glycoprotein has the amino acid sequence shown in SEQ ID NO: 1.

(SEQ ID NO: 1)
KFTIVFPHNQKGNWKNVPSNYHYCPSSSDLNWHNDLIGTAIQVKMPQSHK
AIQADGWMCHASKWVTTCDFRWYGPKYITQSIRSFTPSVEQCKESIEQTK
QGTWLNPGFPPQSCGYATVTDAEAVIVQVTPHHVLVDEYTGEWVDSQFIN
GKCSNYICPTVHNSTTWHSDYKVKGLCDSNLISMDITFFSEDGELSSLGK
EGTGFRSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TTEQELWDDWAPYEDVEIGPNGVLRTSSGYKFPLYMIGHGMLDSDLHLSS
KAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK.

According to the embodiments of the present invention, the single-chain antibody targets a cell-specific antigen.

The single-chain antibody according to the embodiments of the present invention can optionally be set to target any specific antigen, for example, targets tumor cell specific antigen CD19 or Siglec15, and then packages the Obtained virus under the mediation of the targeted binding of single-chain antibodies to cell-specific antigens, the specific targeted binding of the virus to cells (such as tumor cells and immune cells) is realized, thereby the specific infection of the virus to the cells is realized.

According to the embodiments of the present invention, the fusion protein further comprises a first connecting peptide. Thereby, the single-chain antibody region is separated from the C-terminal region of the envelope protein to reduce functional interference between the two.

According to the embodiments of the present invention, the first connecting peptide has the amino acid sequence shown in SEQ ID NO: 2.

AAATTT (SEQ ID NO:2).

According to the embodiments of the present invention, the C-terminal domain of the envelope protein has the amino acid sequence shown in SEQ ID NO: 3.

(SEQ ID NO: 3)
FEHPHIQDAASQLPDDESLFFGDTGLSKNPIELVEGWFSSWKSSIASFFF
IIGLIIGLFLVLRVGIHLCIKLKHTKKRQIYTDIEMNRLGK.

According to the embodiments of the present invention, the fusion protein has the amino acid sequence shown in SEQ ID NO:4 or SEQ ID NO:10.

(SEQ ID NO: 4)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSAAATTTFE
HPHIQDAASQLPDDESLFFGDTGLSKNPIELVEGWFSSWKSSIASFFFII
GLIIGLFLVLRVGIHLCIKLKHTKKRQIYTDIEMNRLGK.
(SEQ ID NO: 10)
DIKMTQSPSSMYASLGERVTITCKASQDINSYLSWFQQKPGKSPKTLIYR
ANRLVDGVPSRFSGSGSGQDYSLTISSLEYEDMGIYYCLQYDEFPYTFGG
GTKLEIKGGGGSGGGGSGGGGSQVQLQQPGAELVKPGASVKMSCKASGYT
FTSYWITWVIQRPGQGLEWIGDIYCGSDTMHYNEKFKNKATLTVDTSSST
AYMQLSSLTSEDSAVYYCARWWDYGSSYDYFDYWGQGTTLTVSSAAATTT
FEHPHIQDAASQLPDDESLFFGDTGLSKNPIELVEGWFSSWKSSIASFFF
IIGLIIGLFLVLRVGIHLCIKLKHTKKRQIYTDIEMNRLGK.

According to the embodiments of the present invention, the viral vectors further comprises: a first promoter, which is operably linked to the first nucleic acid molecule; and a second promoter, which is operably linked to the second nucleic acid molecule. Thereby, the first nucleic acid molecule and the second nucleic acid molecule are respectively under the regulation of the first promoter and the second promoter to achieve high-efficiency expression of the first nucleic acid molecule and the second nucleic acid molecule.

According to the embodiments of the present invention, each of the first promoter and the second promoter is independently selected from CMV, EF-1, and RSV promoters.

According to the embodiments of the present invention, the first nucleic acid

molecule has the nucleotide sequence shown in SEQ ID NO: 5.

(SEQ ID NO: 5)
ATGAAGTGCCTTTTGTACTTAGCCTTTTTATTCATTGGGGTGAATTGCAA
GTTCACCATAGTTTTTCCACACAACCAAAAAGGAAACTGGAAAAATGTTC
CTTCTAATTACCATTATTGCCCGTCAAGCTCAGATTTAAATTGGCATAAT
GACTTAATAGGCACAGCCTTACAAGTCAAAATGCCCCAAAGTCACAAGGC
TATTCAAGCAGACGGTTGGATGTGTCATGCTTCCAAATGGGTCACTACTT
GTGATTTCCGCTGGTATGGACCGAAGTATATAACACATTCCATCCGATCC
TTCACTCCATCTGTAGAACAATGCAAGGAAAGCATTGAACAAACGAAACA
AGGAACTTGGCTGAATCCAGGCTTCCCTCCTCAAAGTTGTGGATATGCAA
CTGTGACGGATGCCGAAGCAGTGATTGTCCAGGTGACTCCTCACCATGTG
CTGGTTGATGAATACACAGGAGAATGGGTTGATTCACAGTTCATCAACGG
AAAATGCAGCAATTACATATGCCCCACTGTCCATAACTCTACAACCTGGC
ATTCTGACTATAAGGTCAAAGGGCTATGTGATTCTAACCTCATTTCCATG
GACATCACCTTCTTCTCAGAGGACGGAGAGCTATCATCCCTGGGAAAGGA
GGGCACAGGGTTCAGAAGTAACTACTTTGCTTATGAAACTGGAGGCAAGG
CCTGCAAAATGCAATACTGCAAGCATTGGGGAGTCAGACTCCCATCAGGT
GTCTGGTTCGAGATGGCTGATAAGGATCTCTTTGCTGCAGCCAGATTCCC
TGAATGCCCAGAAGGGTCAAGTATCTCTGCTCCATCTCAGACCTCAGTGG
ATGTAAGTCTAATTCAGGACGTTGAGAGGATCTTGGATTATTCCCTCTGC
CAAGAAACCTGGAGCAAAATCAGAGCGGGTCTTCCAATCTCTCCAGTGGA
TCTCAGCTATCTTGCTCCTAAAAACCCAGGAACCGGTCCTGCTTTCACCA
TAATCAATGGTACCCTAAAATACTTTGAGACCAGATACATCAGAGTCGAT
ATTGCTGCTCCAATCCTCTCAAGAATGGTCGGAATGATCAGTGGAACTAC
CACAGAACAGGAACTGTGGGATGACTGGGCACCATATGAAGACGTGGAAA
TTGGACCCAATGGAGTTCTGAGGACCAGTTCAGGATATAAGTTTCCTTTA
TACATGATTGGACATGGTATGTTGGACTCCGATCTTCATCTTAGCTCAAA
GGCTCAGGTGTTCGAACATCCTCACATTCAAGACGCTGCTTCGCAACTTC
CTGATGATGAGAGTTTATTTTTTGGTGATACTGGGCTATCCAAAAATCCA
ATCGAGCTTGTAGAAGGTTGGTTCAGTAGTTGGAAAAGCTCTATTGCCTC
TTTTTTCTTTATCATAGGGTTAATCATTGGACTATTCTTGGTTCTCCGAG
TTGGTATCCATCTTTGCATTAAATTAAAGCACACCAAGAAAAGACAGATT
TATACAGACATAGAGATGAACCGACTTGGAAAG.

According to the embodiments of the present invention, the second nucleic acid. molecule has the nucleotide sequence shown in SEQ ID NO:6.

(SEQ ID NO: 6)
ATGAAGTGCCTTTTGTACTTAGCCTTTTTATTCATTGGGGTGAATTGCGA
CATTCAGATGACTCAGACCACCTCCTCCCTGTCCGCCTCCCTGGGCGACC
GCGTGACCATCTCATGCCGCGCCAGCCAGGACATCTCGAAGTACCTCAAC
TGGTACCAGCAGAAGCCCGACGGAACCGTGAAGCTCCTGATCTACCACAC
CTCCCGGCTGCACAGCGGAGTGCCGTCTAGATTCTCGGGTTCGGGGTCGG
GAACTGACTACTCCCTTACTATTTCCAACCTGGAGCAGGAGGATATTGCC
ACCTACTTCTGCCAACAAGGAAACACCCTGCCGTACACTTTTGGCGGGGG
AACCAAGCTGGAAATCACTGGGGGCGGAGGATCCGGTGGAGGCGGAAGCG
GGGGTGGAGGATCCGAAGTCAAGCTGCAGGAATCAGGACCTGGCCTGGTG
GCCCCGAGCCAGTCACTGTCCGTGACTTGTACTGTGTCCGGAGTGTCGCT
CCCGGATTACGGAGTGTCCTGGATCAGGCAGCCACCTCGGAAAGGATTGG
AATGGCTCGGAGTCATCTGGGGTTCCGAAACCACCTATTACAACTCGGCA
CTGAAATCCAGGCTCACCATTATCAAGGATAACTCCAAGTCACAAGTGTT
CCTGAAGATGAATAGCCTGCAGACTGACGACACGGCGATCTACTATTGCG
CCAAGCACTACTACTACGGCGGATCCTACGCTATGGACTACTGGGGCCAG
GGGACCAGCGTGACCGTGTCATCCGCGGCCGCAACTACCACCTTCGAACA
TCCTCACATTCAAGACGCTGCTTCGCAACTTCCTGATGATGAGAGTTTAT
TTTTTGGTGATACTGGGCTATCCAAAAATCCAATCGAGCTTGTAGAAGGT
TGGTTCAGTAGTTGGAAAAGCTCTATTGCCTCTTTTTTCTTTATCATAGG
GTTAATCATTGGACTATTCTTGGTTCTCCGAGTTGGTATCCATCTTTGCA
TTAAATTAAAGCACACCAAGAAAAGACAGATTTATACAGACATAGAGATG
AACCGACTTGGAAAG.

According to the embodiments of the present invention, the first viral vector and the second viral vector are the same vector:

According to the embodiments of the present invention, the first viral vector and the second viral vector are the same vector, the vector further comprises: an internal ribosome entry site sequence (IRES), wherein the internal ribosome entry site sequence is arranged between the first nucleic acid molecule and the second nucleic acid molecule. The expression of the two proteins before and after the internal ribosome entry site is usually proportional. The introduction of the internal ribosome entry site sequence allows the first nucleic acid molecule and the second nucleic acid molecule to be independently translated and expressed, and the resulting envelope protein and fusion protein are in a non-fusion form. The introduction of the internal ribosome entry site sequence effectively guarantees the biological effects of the envelope protein and the fusion protein, so that the specific binding adsorption and infectivity of the virus obtained by packaging is remarkable, and the virus titer is high.

According to the embodiments of the present invention, the first viral vector and the second viral vector are the same vector, the vector further comprises: a third nucleic acid molecule, which is arranged between the first nucleic acid molecule and the second nucleic acid molecule, and the third nucleic acid molecule encodes a second connecting peptide, and the second connecting peptide can be cleaved. The introduction of the third nucleic acid molecule makes the expression of the envelope protein and the fusion protein in a non-fusion form, thereby ensuring the biological function of the envelope protein and the fusion protein, so that the specific binding adsorption and infection ability of the virus obtained by packaging is remarkable, and the virus titer is high.

According to the embodiments of the present invention, the first viral vector and the second viral vector are the same vector, the ratio of the copy number of the first nucleic acid molecule and the second nucleic acid molecule is 1:1˜4:1. It should be noted that the “ratio of the copy number of the first nucleic acid molecule and the second nucleic acid molecule” herein refers to the ratio of the number of the first nucleic acid molecule and the second nucleic acid molecule carried on the vector when the first viral vector and the second viral vector are the same vector, that is, when the first nucleic acid molecule and the second nucleic acid molecule are the same vector, so as to ensure that the ratio of the protein expression amount of the first nucleic acid molecule and the second nucleic acid molecule is approximately the same. The inventors found that when the ratio of the number of the first nucleic acid molecule and the second nucleic acid molecule carried on the vector is 1:1˜4:1, the virus titer and the infection efficiency of the virus are both higher.

According to the embodiments of the present invention, the ratio of the copy number of the first nucleic acid molecule and the second nucleic acid molecule is 2:1 to 4:1. According to the specific embodiments of the present invention, when the ratio of the number of the first nucleic acid molecule and the second nucleic acid molecule carried on the vector is 2:1 to 4:1, the virus titer and the infection efficiency of the virus are both further improved.

According to the embodiments of the present invention, the ratio of the copy number of the first nucleic acid molecule and the second nucleic acid molecule is 2:1. The inventors found that when the ratio of the number of the first nucleic acid molecule and the second nucleic acid molecule carried on the vector is 2:1, the virus titer and the infection efficiency of the virus will reach an optimal balance.

According to the embodiments of the present invention, the first viral vector and the second viral vector are pMD2.G, pCMV, pMD2.G mutant or pCMV mutant. According to the embodiments of the present invention, the types of the first viral vector and the second viral vector are not particularly limited. A vector that can express VSV-G or a vector that can express a VSV-G or a VSV-G mutant can be used.

According to the embodiments of the present invention, the viral vectors further comprises: a third viral vector and a fourth viral vector, the third viral vector carries the gene of interest, and the fourth viral vector carries the viral structural protein gene and viral packaging enzyme gene and optional regulatory factor rev gene.

According to the embodiments of the present invention, the structural protein gene, the viral packaging enzyme gene and the regulatory factor rev gene are arranged on the same fourth viral vector or different fourth viral vectors. For example, the expression products of the lentiviral vector psPAX2 include structural protein gag, packaging enzyme pol (including reverse transcriptase, protease and integrase) and regulatory factor rev, wherein, the regulatory factor rev can increase the product titer to a certain extent, but it is not necessary for lentiviral packaging. Rev (pRSV-rev) and gag-pol (pMDLg-pRRE) can be divided into two plasmids for expression; or rev (pRSV-rev), gag (pCMV-gag), pol (pCMV-gag) can be divided into three plasmids for expression.

According to the embodiments of the present invention, the viral packaging enzyme comprises at least one of reverse transcriptase, protease, and integrase.

According to the embodiments of the present invention, the third viral vector is a transfer vector.

According to the embodiments of the present invention, the transfer vector contains a lentivirus packaging signal.

According to the embodiments of the present invention, the lentivirus packaging signal comprises: Ψ.

According to the embodiments of the present invention, the transfer vector is a pLV vector.

According to the embodiments of the present invention, the fourth viral vector is psPAX2.

In the other aspect, provided herein is a method for obtaining lentivirus. According to the embodiments of the present invention, the method comprises: introducing the group of viral vectors provided herein into a first recipient cell; culturing the first recipient cell to obtain a virus. The lentivirus obtained by the method has a high titer, and the abilities to target binding and infect cells are significantly improved.

According to the embodiments of the present invention, the method provided herein may further comprise at least one of the following additional technical features:

according to the embodiments of the present invention, the virus is lentivirus, the first viral vector and the second viral vector are different vectors, the mass ratio of the third viral vector, the fourth viral vector, the first viral vector and the second viral vector is 2:1:0.25˜2:1:1:1

According to the ratio of viral vectors of the embodiments of the present invention, the lentivirus titer and lentivirus infection efficiency are both high.

According to the embodiments of the present invention, the mass ratio of the third viral vector, the fourth viral vector, the first viral vector and the second viral vector is 2:1:1:0.5. The inventors found that when the mass ratio of the third viral vector, the fourth viral vector, the first viral vector and the second viral vector is 2:1:1:0.5, the titer and the infection efficiency of the lentivirus will reach an optimal balance.

According to the embodiments of the present invention, the first recipient cell is 293T.

In the third aspect, provided herein is a lentivirus. According to the embodiments of the present invention, the lentivirus is obtained by packaging as described herein. The lentivirus has a high titer and has the abilities to target binding and infect cells.

In the fourth aspect, provided herein is a lentivirus. According to the embodiments of the present invention, the lentivirus expresses an envelope protein and a fusion protein, the fusion protein comprises a single-chain antibody and the C-terminal domain of the envelope protein, and the C-terminal domain of the envelope protein comprises the transmembrane and intracellular regions of the envelope protein, the C-terminus of the single-chain antibody is connected to the N-terminus of the C-terminal domain of the envelope protein. The lentivirus provided herein has a high titer and has the abilities to target binding and infect cells.

According to the embodiments of the present invention, the envelope protein is an envelope G glycoprotein or a mutant of envelope G glycoprotein of vesicular stomatitis virus.

in the fifth aspect, provided herein is a pharmaceutical composition. According to the embodiments of the present invention, the pharmaceutical composition comprises the group of viral vectors or the lentivirus provided herein, and the viral vectors or lentivirus carries the gene of interest. It should be noted that the “gene of interest” herein refers to the exogenous gene that the viral vectors or lentivirus needs to carry. After the virus infects the cell, the exogenous gene enters the target cell, and the expression of the gene of interest is achieved in the target cell. When the expression of the gene of interest in the target cell can achieve direct or indirect therapeutic effects, the pharmaceutical composition formed by the viral vectors or lentivirus achieves the effect of bringing the gene of interest into the targeted cell, thereby achieving the targeted treatment of diseases.

In the sixth aspect, provided herein is a method for expressing gene of interest. According to the embodiments of the present invention ; the method comprises: introducing the viral vectors or lentivirus integrated with the gene of interest into the second recipient cell; culturing the second recipient cell to express the gene of interest. According to the embodiments of the present invention, the expression of the gene of interest in the recipient cell is effectively realized. For example, in the embodiments of the present invention, mcherry or BFP is a gene of interest that can be carried, in order to verify the feasibility of the targeting vector platform, the inventor used mcherry gene or BFP gene as a tag gene to express fluorescent protein, thereby characterizing the positive rate of virus infection in the recipient cell.

According to the embodiments of the present invention, the method may further comprise at least one of the following additional technical features:

according to the embodiments of the present invention, the introduction into the second recipient cell is carried out by electrotransfection, transfection or infection. It should be noted that the “electrotransfection” or “transfection” refers to a method of introducing the viral vectors into a recipient cell, and the “infection” refers to a process in which the virus actively binds and fuses with the cell membrane to enter the cell. Wherein, “electrotransfection” refers to a method of introducing vectors for virus packaging into recipient cell by means of electrical stimulation, and “transfection” refers to a method of introducing vectors for virus packaging into recipient cell through chemical mediators, such as liposomes.

According to the embodiments of the present invention, the second recipient cell is a somatic cell.

According to the embodiments of the present invention, the second recipient cell is a tumor cell. When the expression of the gene of interest in tumor cells can achieve direct or indirect therapeutic effects, the method provided herein achieves specific killing of tumor cells and achieves specific treatment of tumors.

According to the embodiments of the present invention, the “pMD2.G mutant” or “pMD2.G-Mut” refers to a plasmid (VSV-G-K47Q\354Q) expressing lentiviral envelope protein containing K47Q\354Q mutation site.

### DESCRIPTION OF THE DRAWINGS

FIG. 1 is a structural pattern diagram of the envelope fusion protein of targeting CD19 antigen (antiCD19-G) according to the embodiments of the present invention; wherein anti-CD19 say represents the variable region sequence of the CD19 single-chain antibody, and VSV-G C-terminal represents the C-terminal domain of the lentiviral envelope protein;

FIG. 2 is a map of pMD2.G mutant (pMD2.G-Mut) according to the embodiments of the present invention;

FIG. 3 is a titer result graph of CD19-targeting lentivirus obtained by packaging pMD2.G-Mut and pMD2.antiCD19-G in different proportions according to the embodiments of the present invention, wherein the abscissa represents the type of lentivirus, the types and ratios of vectors contained in each lentivirus are shown in Table 1; the ordinate represents the lentivirus titer measured by the lentivirus infecting HEK-293T-CD19 cells;

FIG. 4 is a titer result graph of CD19-targeting lentivirus obtained by packaging according to the embodiments of the present invention, wherein the abscissa represents the type of lentivirus, the types and ratios of vectors contained in each lentivirus are shown in Table 2; the ordinate represents the lentivirus titer measured by the lentivirus infecting HEK-293T cells;

FIG. 5 is a flow analysis diagram of 293T infected by CD19-targeting lentivirus obtained by packaging according to the embodiments of the present invention, wherein CD19ta is a scFv that can bind to CD19;

FIG. 6 is a capability analysis diagram of 293T infected by CD19-targeting lentivirus obtained by packaging according to the embodiments of the present invention, wherein the abscissa represents the type of lentivirus, the types and ratios of vectors contained in each lentivirus are shown in Table 2; the ordinate represents that the ratio of mCherry positive rate of HEK-293T-CD19 cells and HEK-293T cells after the mixed system of HEK-293T-CD19 and HEK-293T cells is infected by lentivirus, CD19ta is a scFv that can bind to CD19;

FIG. 7 is a capability analysis diagram of 293T cells infected by Siglec15-targeting lentivirus obtained by packaging according to the embodiments of the present invention, wherein the abscissa represents the type of the lentivirus, the types and ratios of vectors contained in each lentivirus are shown in Table 3; the ordinate represents the ratio of the BFP positive rate of HEK-293T-HS15 cells and HEK-293T cells after the mixed system of HEK-293T-HS15 and HEK-293T cells is infected by the lentivirus, HEK-293T-HS15 represents HEK 293T cells stably expressing human Siglec15 protein, and 5G12 represents scFv that capable of binding to Siglec15.

### EXAMPLES

Embodiments of the present invention are described in detail below, and examples of the embodiments are shown in drawings.

According to specific embodiments of the present invention, provided herein is a group of novel lentiviral vectors with targeted transfection ability, the group of vectors comprise a coding region carrying an envelope protein VSV-G or VSV-G mutant, and a coding region carrying a fusion protein obtained by linking the single-chain antibody scFv and C-terminal domain of VSV-G through a connecting peptide.

The lentiviral vectors provided herein have the following characteristics: \textcircled{1}VSV-G mutant is a mutant that weakens the ability of VSV-G to adsorb target cells, but retains the ability of cell membrane fusion; \textcircled{2}scFv can be one or more in series; \textcircled{3}the C-terminal domain of VSV-G at least includes the intracellular and transmembrane regions of VSV-G; \textcircled{4}a viral vector can contain one or more fusion proteins.

The advantages of this study: \textcircled{1}the fusion protein formed by scFv and the C-terminal domain of VSV-G has the same transmembrane and intracellular regions as VSV-G, so that the fusion protein still maintains the interaction with the matrix protein, making it efficiently assembled on the envelope of the lentiviral particle without interfering with the budding of the virus particle, thereby not having a significant impact on virus titer. \textcircled{2}The envelope of the lentiviral particle still contains intact VSV-G or its mutant, which can maintain the stability of the virus particle. \textcircled{3}Through the binding of scFv to the corresponding antigen, the lentiviral particle can actively infect target cells which expresses corresponding antigens, and increase the infection efficiency of the target cells under the same infection multiplicity conditions. \textcircled{4}The scFv has a clear functional background and guaranteed safety, and any antigen can be screened to specifically bind scFv, making the virus packaged by the lentiviral vectors can be universally applied to the targeted infection of various types of cells. \textcircled{5}VSV-G is replaced with a mutant with weakened ability to adsorb target cells, so that the specific binding force between scFv and the corresponding antigen dominates the process of viral particle adsorption, and further improves the targeting of recombinant lentivirus.

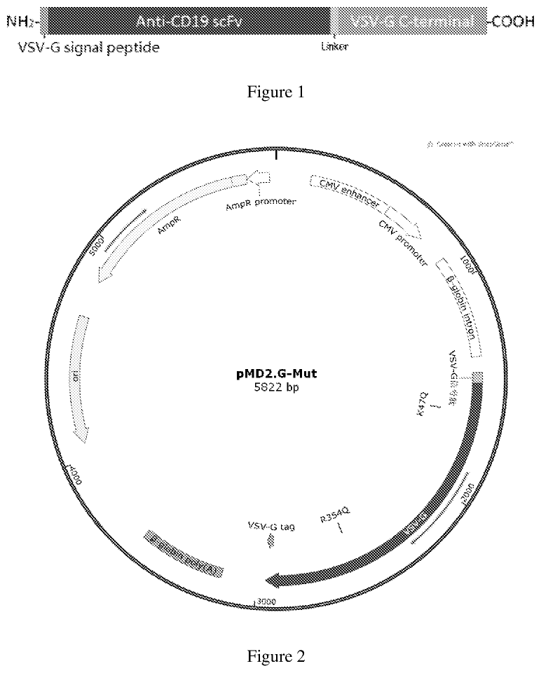

According to specific embodiments of the present invention, provided herein is a method for constructing and using a new type of lentiviral vectors with the ability to target gene transfection, comprising the following steps (taking the lentiviral vector targeted for transfection of CD19 ⁺ cells as an example):
1. Designing a fusion protein (CD19 scFv-VSV-G-CT (antiCD19-G for short)) formed by a single-chain antibody targeting CD19 and the C-terminal domain of VSV-G The structural pattern diagram is shown in FIG. 1. Wherein, the VSV-G signal peptide is located at the amino terminus of the VSV-G precursor protein and it is the membrane-localized telopeptide of the VSV-G protein. The VSV-G signal peptide helps the VSV-G protein to locate to the endoplasmic reticulum, after the protein matures, it is hydrolyzed and separated. The VSV-G protein of the viral particle does not contain this signal peptide.

The representative amino acid sequence of the signal peptide is shown in SEQ ID NO:7.

(SEQ ID NO: 7)
MKCLLYLAFLFIGVNC.

The amino acid sequence of the anti-CD19 scFv is shown in SEQ ID NO:8.

(SEQ ID NO: 8)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSS.

The amino acid sequence of the first linker peptide (Linker) is shown in SEQ ID NO:2.

(SEQ ID NO: 2)
AAATTT.

VSV-G C-Terminal contains amino acids 405-495 (91 in total) of VSV-G protein, and its amino acid sequence is:

(SEQ ID NO: 3)
FEHPHIQDAASQLPDDESLFFGDTGLSKNPIELVEGWFSSWKSSIASFFF
IGLIIGLFLVLRVGIHLCIKLKHTKKRQIYTDIEMNRLGK.

2. Constructing CG Fusion Membrane Protein Expression Plasmid

The DNA sequence in the model of VSV-G signal peptide-scFv-Linker-(VSV-G-CT) is designed and entrusted gene synthesis company to synthesize. The pMD2.G plasmid (VSV-G protein expression plasmid) is used as a vector to construct the fusion protein expression plasmid pMD2. antiCD19-G.
3. Constructing VSV-G Weak Adsorption Mutants (Reference: Structural Basis for the Recognition of LDL-Receptor Family Members by VSV Glycoprotein, Nature Communications. 2018)

The DNA sequence of the K47Q and R354Q double-point mutant is designed and entrusted gene synthesis company to synthesize. The VSV-G expression plasmid pMD2.G is used as a vector to construct the VSV-G-K47Q\354Q expression plasmid pMD2.G-Mut. Studies have found that K47Q and R354Q mutations can only reduce the membrane adsorption capacity of VSV-G, but not affect the membrane fusion capacity of VSV-G.
4. Lentivirus Packaging and Harvest

\textcircled{1}The lentiviral vector packaging plasmids were co-transfected into 293T cells.

\textcircled{2}The virus-containing culture supernatant is collected after 48-72 h of transfection; filtered with 0.45μm aliquoted and stored in an ultra-low temperature refrigerator.
5. Detection and Analysis of Targeted Transfection Ability of Lentiviral Vectors

Lentiviral vectors are used to infect the mixed cultured cells of CD19⁺ and CD19⁻, and the positive rates of the two types of cells are compared.

\textcircled{2}Under the same infection conditions, the positive rate of infected CD19 ⁺ cells/the positive rate of infected CD19⁺ cells can be used to evaluate the ability of the lentivirus to target infect CD19⁺ cells (targeted infectivity for short). When the targeted infectivity ≤1, the lentivirus has no ability to target CD19⁺ cells; when the targeted infectivity >1, the lentivirus has the ability to target CD19⁺ cells, and the larger the value, the stronger the ability to target CD19⁺ cells.

Embodiments of the present invention are described in detail below, and examples of the embodiments are shown in drawings. The embodiments described below with reference to the drawings are exemplary and are intended to explain the present invention and should not be construed as limiting the present invention.

it should be noted that the “plasmid” and “vector” described in the following embodiments have the same meaning and can be used interchangeably.

### EXAMPLE 1

1. Packaging a series of lentiviruses with different antiCD19-G content targeted to infect CD19⁺ cells

1.1.1 Lentivirus Packaging Information

According to the plasmids and mass ratios shown in Table 1 (to maintain the same mass of pLV-mCherry and psPAX2 plasmids between each group), 293T cells were co-transfected to package the lentivirus.

TABLE 1

| lentivirus | Names of lentiviral vectors and ratios |
|---|---|
| LVm-CD19ta(1:0.25) | pLV-mCherry:psPAX2:pMD2.G-Mut:pMD2. |
| | antiCD19-G = 2:1:1:0.25 |
| LVm-CD19ta(1:0.5) | pLV-mCherry:psPAX2:pMD2.G-Mut:pMD2. |
| | antiCD19-G = 2:1:1:0.5 |
| LVm-CD19ta(1:1) | pLV-mCherry:psPAX2:pMD2.G-Mut:pMD2. |
| | antiCD19-G = 2:1:1:1 |
| LVm-CD19ta(1:2) | pLV-mCherry:psPAX2:pMD2.G-Mut:pMD2. |
| | antiCD19-G = 2:1:1:2 |
| LVm-CD19ta(1:4) | pLV-mCherry:psPAX2:pMD2.G-Mut:pMD2. |
| | antiCD19-G = 2:1:1:4 |
| LVm-CD19ta(1:8) | pLV-mCherry:psPAX2:pMD2.G-Mut:pMD2. |
| | antiCD19-G = 2:1:1:8 |

Wherein, pLV-mCherry is a transfer plasmid carrying mCherry sequence, the gene of interest is the mCherry sequence, and psPAX2 is a plasmid expressing lentiviral structural proteins gag and pol (reverse transcriptase, protease, integrase, packaged into viral particle, non-structural protein), pMD2.G-Mut is a plasmid expressing lentiviral membrane protein mutants (VSV-G-K47Q\R354Q), pMD2. antiCD19-G is a plasmid expressing a chimeric protein containing a say targeting CD19 and the C-terminal domain of the VSV-G protein, wherein the map of the pMD2.G mutant used in this example is shown in FIG. 2.

1.1.2 Evaluation of Lentiviral Vector Yield

1) Evaluation of Cell Line Construction:

A 293T cell line (HEK-293T-CD19) that stably co-expressed CD19 and GFP was constructed. The purpose of expressing GFP was to use GFP positive to indicate CD19 positive cells.

2) Evaluation of Lentivirus Packaging, Harvesting and Yield

According to the experimental group set up in Table 1, 293T cells were co-transfected for 48-72 h, and then the virus liquids of each group were collected. The virus liquids of each group were filtered through a 0.45 μm sterile filter. 200 μL of HEK-293T-CD19 cells was spread into the wells of a 24-well plate. The filtered lentivirus was diluted 100 times, then 200 μL/well of the solution was added to the corresponding cell well, and mixed gently and thoroughly, the plate was incubated at 37° C. and 5% CO₂ for 34 days. Flow cytometry was used to detect the percentage of mCherry⁺ cells in the infected cells.

3) Results and Analysis

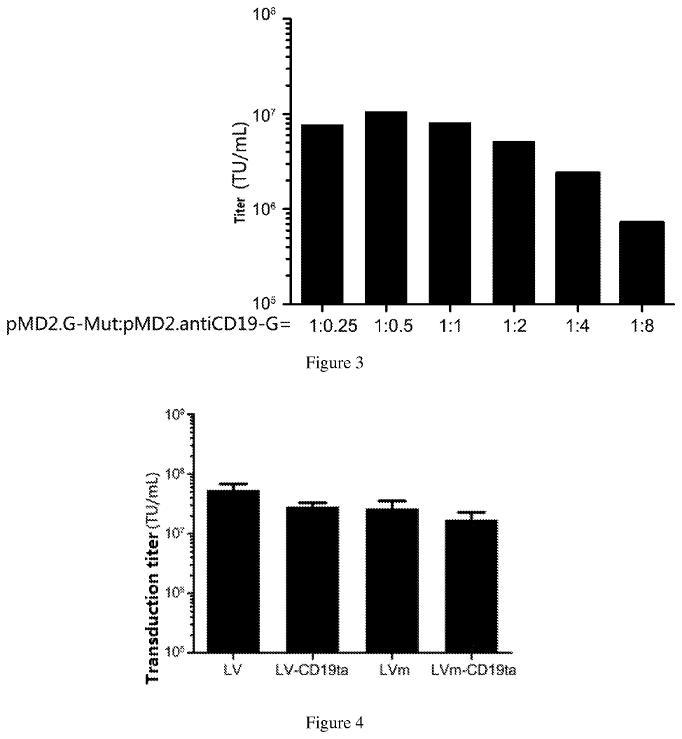

When the mass ratio of plasmids expressing VSV-G-1(47Q\R354Q protein and antiCD19-G fusion protein is 1:0.25˜1:1 (i.e. 4:1˜1:1), the virus titer is higher. Wherein 1:0.5 (i.e. 2:1) is the highest (the result is shown in FIG. 3). Virus yield=virus titer×virus liquid volume. When the harvested virus liquid volume is the same, the level of virus titer represents the level of virus yield, that is, when the mass ratio of plasmids expressing VSV-G-K47Q\R354Q protein and antiCD19-G fusion protein is 1:0.25˜1:1 (i.e. 4:1˜1:1), the virus yield is higher, and 1:0.5 is the highest.

1.2 Packaging a Series of Lentiviruses Targeted to Infect CD19⁺ Cells.

1.2.1 Lentivirus Packaging Information

According to the plasmid and ratio in Table 2 below (to maintain the same amount of pLV-mCherry and psPAX2 plasmids between each group), 293T cells were co-transfected to package lentivirus.

TABLE 2

| lentivirus | Names of lentiviral vectors and ratios |
|---|---|
| Negative Control(NC) | pLV-mCherry:psPAX2 = 2:1 |
| LV (Lentivirus) | pLV-mCherry:psPAX2:pMD2.G = 2:1:1 |
| LV-CD19ta | pLV-mCherry:psPAX2:pMD2.G:pMD2. |
| | antiCD19-G = 2:1:1:0.5 |
| LVm | pLV-mCherry:psPAX2:pMD2.G-Mut = 2:1:1 |
| LVm-CD19ta | pLV-mCherry:psPAX2:pMD2.G-Mut:pMD2. |
| | antiCD19-G = 2:1:1:0.5 |

Wherein, pLV-mCherry is a transfer plasmid carrying mCherry sequence, the gene of interest is the mCherry sequence, and psPAX2 is a plasmid expressing lentiviral structural proteins gag and poi (reverse transcriptase, protease, integrase, packaged into viral particle, non-structural protein). pMD2.G is a plasmid expressing lentiviral envelope protein (expressing VSV-G), pMD2.G-Mut is a plasmid expressing lentiviral membrane protein mutant (VSV-G-K47Q\R354Q, pMD2. antiCD19-G is a plasmid expressing a chimeric protein containing a scFv targeting CD19 and the C-terminal domain of the VSV-G protein, wherein the map of the pMD2.G mutant used in this example is shown in FIG. 2.

1.2.2 Evaluation of the Targeting Performance of Lentiviral Vector Targeting CD19⁺ Cells

1) Evaluation of Cell Line Construction:

A 293T cell line (HEK-293T-CD19) that stably co-expresses CD19 and GFP was constructed. The purpose of expressing GFP was to use GFP positive to indicate CD19 positive cell.

2) Lentivirus Packaging, Harvest, Titer Determination and Target Cell Transfection

According to the experimental group set up in Table 2, 293T were co-transfected for 48-72 h. The virus liquids of each group were collected and stored in ultra-low temperature refrigerator (<−75″C). The titer was measured with 293T cells.

293T and HEK-293T-CD19 cells were mixed at a ratio of 1:1 and spread into a 24-well plate. The frozen lentivirus solution was diluted 100 times, and 200 μLL/well of the solution was added to the corresponding cell wells, mixed gently and thoroughly. (There are two purposes for mixed transfection of the two types of cells: a. To ensure that the transfection conditions of the two cells are completely the same; b. To simulate the transfection conditions when target cells and non-target cells coexist in in vivo gene therapy.)

The plate was incubated at 37° C. and 5% CO₂ for 3-4 days. Flow cytometry was used to detect the percentage of mCherry⁺ cells and the percentage of GFP-positive (GFP⁺) cells in the infected cells.

3) Results and Analysis

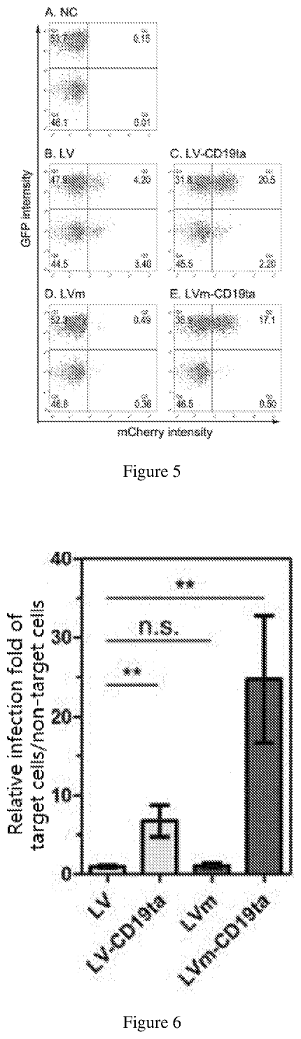

The expression of antiCD19-G fusion protein will not cause a significant drop in the production titer of lentivirus (results are shown in FIG. 4), and the titer can reach more than 10⁷, which is significantly better than that of the targeted virus in the prior art. The expression of antiCD19-G fusion protein also can enhance the infectivity of lentivirus to CD19⁺ cells (results are shown in FIG. 5 and FIG. 6).

The expression of mutant envelope protein (VSV-G-K47Q\R354Q) can reduce the infectivity of lentivirus (results are shown in FIG. 5D and FIG. 5B), and the expression of antiCD19-G fusion protein specifically enhances the infectivity of lentivirus to target CD19⁺ cells (results are shown in FIG. 5E), so that LVm-CD19ta have a stronger infectivity to target CD19⁺ cells than LV-CD19ta (results are shown in FIG. 6).

### EXAMPLE 2

2.1 Packaging a Series of Lentiviruses Targeted to Infect Human Siglec15 ⁺ Cells

According to the plasmids and ratios in Table 3 below (to maintain the same amount of pLV-BFP and psPAX2 plasmids between each group), HEK 293T cells were co-transfected to package lentivirus.

TABLE 3

| lentivirus | Names of lentiviral vectors and ratios |
|---|---|
| LV-BFP | pLV-BFP:psPAX2:pMD2.G = 2:1:1 |
| LV-BFP-5G12 | pLV-BFP:psPAX2:pMD2.G:pMD2. antiSiglec15-G = |
| | 2:1:1:0.5 |
| LVm-BFP | pLV-BFP:psPAX2:pMD2.G-Mut = 2:1:1 |
| LVm-BFP-5G12 | pLV-BFP:psPAX2:pMD2.G-Mut:pMD2. |
| | antiSiglec15-G = 2:1:1:0.5 |

Wherein pLV-BFP is a transfer plasmid carrying the BFP (Blue Fluorescent Protein) sequence, and the gene of interest is the BFP sequence. psPAX2 is a plasmid expressing lentiviral structural protein gag and pol (reverse transcriptase, protease, integrase, packed into viral particles, non-structural protein), pMD2.G is a plasmid expressing lentiviral envelope protein (VSV-G), pMD2.G-Mut is a plasmid expressing lentiviral membrane protein mutant (VSV-G-K47Q\R354Q), antiSiglec15-G is a plasmid expressing chimeric protein containing the scFv targeting Siglec15 and the C-terminal domain of the VSV-G protein. The map of the pMD2.G mutant used in this example is shown in FIG. 2.

The amino acid sequence of anti-Siglec15 scFv is shown in SEQ ID NO: 9:

(SEQ ID NO: 9)
DIKMTQSPSSMYASLGERVTITCKASQDINSYLSWFQQKPGKSPKTLIYR
ANRLVDGVPSRFSGSGSGQDYSLTISSLEYEDMGIYYCLQYDEFPYTFGG
GTKLEIKGGGGSGGGGSGGGGSQVQLQQPGAELVKPGASVKMSCKASGYT
FTSYWITWVIQRPGQGLEWIGDIYCGSDTMHYNEKFKNKATLTVDTSSST
AYMQLSSLTSEDSAVYYCARWWDYGSSYDYFDYWGQGTTLTVSS

2.2 Evaluation of the Targeting Performance of Lentiviral Vector Targeting Siglec15′ Cells

1) Evaluation of Cell Line Construction:

A 293T cell line that stably co-expresses Siglec15 and BFP (293T-Siglec15⁺) was constructed.

2) Lentivirus Packaging, Harvest, Titer Determination and Target Cell Transfection

According to the experimental group set up in Table 2, 293T cells were co-transfected for 48-72 h, the virus liquids of each group were collected and stored in ultra-low temperature refrigerator (<−75° C.). The titer was measured with 293T cells.

293T and 293T-Siglec15⁺ cells were mixed at a ratio of 1:1 and spread into a 24-well plate. The frozen lentivirus solution was diluted 100 times, and 200 μL/well of the solution was added to the corresponding cell wells, mixed gently and thoroughly.

The mixed-transfected two kinds of cells were stood and cultured for 3-4 days at 37° C., 5% CO2. Flow cytometry was used to detect the percentage of BFP⁺ cells and Siglec15 positive (Siglec15⁺) cells in the infected cells.

4) Results and Analysis

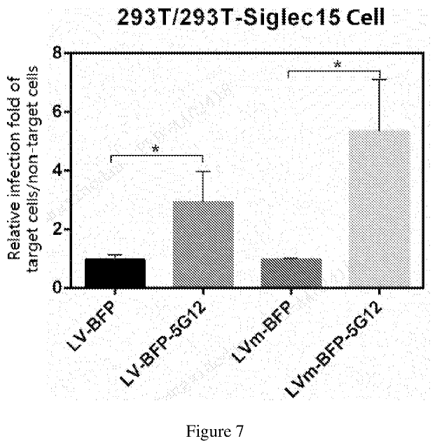

The specific experimental results are shown in FIG. 7. The specifically expression of antiSiglec15-G fusion protein enhances the ability of lentivirus to infect Siglec15⁺ cells, making LV-BFP-5G12 have stronger targeting ability than LV-BFP, and LVm-BFP-5G12 have stronger targeting ability than LVm-BFP.

In addition, the terms “first” and “second” are only used for descriptive purposes, and cannot be understood as indicating or implying relative importance or implicitly indicating the number of indicated technical features. Therefore, the features defined with “first” or “second” may explicitly or implicitly include at least one of the features. In the description of the present invention, “more” means at least two, such as two, three, etc., unless otherwise specifically defined.

Reference throughout this specification to “an embodiment,” “some embodiments,” “one embodiment”, “another example,” “an example,” “a specific examples,” or “some examples,” means that a particular feature, structure, material, or characteristic described in connection with the embodiment or example is included in at least one embodiment or example of the present disclosure. Thus, the appearances of the above terms throughout this specification are not necessarily referring to the same embodiment or example of the present disclosure. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1. A group of viral vectors, comprising:
a first viral vector, wherein the first viral vector carries a first nucleic acid molecule, and the first nucleic acid molecule encodes an envelope protein;
at least a second viral vector, wherein the second viral vector carries a second nucleic acid molecule, the second nucleic acid molecule encodes at least one fusion protein, the fusion protein includes at least one single-chain antibody and the C-terminal domain of the envelope protein, the C-terminal domain of the envelope protein includes a transmembrane region and a intracellular region of the envelope protein, the C-terminus of the at least one single-chain antibody connects with the N-terminus of the C-terminal domain of the envelope protein, the single-chain antibody targets a specific antigen;
the first nucleic acid molecule and the second nucleic acid molecule are arranged to express the envelope protein and the fusion protein, and the envelope protein and the fusion protein are in a non-fusion form.

2. The viral vectors according to claim 1, wherein the viral vectors are retrovirus vectors, lentivirus vectors or other enveloped virus vectors.

3. The viral vectors according to claim 1, wherein the enveloped virus comprises at least one selected from: Bornaviridae, Nyamaviridae, Arenaviridae, Filoviridae, Hantaviridae, Nairoviridae, Orthomyxoviridae, Paramyxoviridae, Bunyaviridae, Phenuiviridae, Rhabdoviridae, Arteriviridae, Coronaviridae, Flaviviridae, Togaviridae, Hepadnaviridae, Spumavirus, Iridoviridae, Herpesviridae, Poxviridae, and Deltavirus;
optionally, the envelope protein is an envelope G glycoprotein or a mutant of envelope G glycoprotein of vesicular stomatitis virus of the Rhabdoviridae.

4. The viral vectors according to claim 3, wherein the mutant of the envelope G glycoprotein has K47Q and R354Q mutations;
optionally, the mutant of the envelope G glycoprotein has the amino acid sequence shown in SEQ ID NO:1.

5. The viral vectors according to claim 1, wherein the single-chain antibody targets a cell-specific antigen.

6. The viral vectors according to claim 1, wherein the fusion protein further comprises a first connecting peptide;
optionally, the first connecting peptide has the amino acid sequence shown in SEQ ID NO: 2;
optionally, the C-terminal domain of the envelope protein has the amino acid sequence shown in SEQ ID NO: 3;
optionally, the fusion protein has the amino acid sequence shown in SEQ ID NO:4 or SEQ ID NO:10.

7. The viral vectors according to claim 1, further comprising:
a first promoter, which is operably linked to the first nucleic acid molecule; and
a second promoter, which is operably linked to the second nucleic acid molecule.

8. The viral vectors according to claim 7, wherein each of the first promoter and the second promoter is independently selected from CMV, EF-1, and RSV promoters.

9. The viral vectors according to claim 1, wherein the first nucleic acid molecule has the nucleotide sequence shown in SEQ ID NO: 5;
optionally, the second nucleic acid molecule has the nucleotide sequence shown in SEQ ID NO:6.

10. The viral vectors according to claim 1, wherein the first viral vector and the second viral vector are the same vector.

11. The viral vectors according to claim 10, further comprising:
an internal ribosome entry site sequence, and the internal ribosome entry site sequence is arranged between the first nucleic acid molecule and the second nucleic acid molecule.

12. The viral vectors according to claim 10, further comprising:
a third nucleic acid molecule, which is arranged between the first nucleic acid molecule and the second nucleic acid molecule, and the third nucleic acid molecule encodes a second connecting peptide, and the second connecting peptide can be cleaved.

13. The viral vectors according to claim 10, wherein the ratio of the copy number of the first nucleic acid molecule and the second nucleic acid molecule is 1:1˜4:1,
optionally, the ratio of the copy number of the first nucleic acid molecule and the second nucleic acid molecule is 2:1˜4:1,
preferably, the ratio of the copy number of the first nucleic acid molecule and the second nucleic acid molecule is 2:1.

14. The viral vectors according to claim 1, wherein the first viral vector and the second viral vector are pMD2.G, pCMV, pMD2.G mutant or pCMV mutant.

15. The viral vectors according to claim 1, further comprising: a third viral vector and a fourth viral vector, the third viral vector carries the gene of interest, and the fourth viral vector carries the viral structural protein gene and viral packaging enzyme gene and optional regulatory factor rev gene;
optionally, the structural protein gene, the viral packaging enzyme gene and the regulatory factor rev gene are arranged on the same fourth viral vector or different fourth viral vectors;
optionally, the viral packaging enzyme comprises at least one of reverse transcriptase, protease, and integrase.

16. The viral vectors according to claim 15, wherein the third viral vector is a transfer vector, and the transfer vector contains a lentiviral packaging signal, optionally, the lentiviral packaging signal comprises: Ψ;
optionally, the transfer vector is pLV;
optionally, the fourth viral vector is psPAX2.

17. A method for obtaining lentivirus, comprising:
introducing the viral vectors according to claim 1 into a first recipient cell; culturing the first recipient cell to obtain a virus.

18. The method according to claim 17, wherein the virus is lentivirus, the first viral vector and the second viral vector are different vectors, the mass ratio of the third viral vector, the fourth viral vector, the first viral vector and the second viral vector is 2:1:1:0.25˜2:1:1:1,
preferably, the mass ratio of the third viral vector, the fourth viral vector, the first viral vector and the second viral vector is 2:1:1:0.5;
optionally, the first recipient cell is 293T.

19. A lentivirus, which is obtained by packaging according to the method of claim 17.

20. A lentivirus, which expresses an envelope protein and a fusion protein, wherein the fusion protein comprises a single-chain antibody and a C-terminal domain of the envelope protein, the C-terminal domain of the envelope protein comprises transmembrane and intracellular regions of the envelope protein, the C-terminus of the single-chain antibody is connected to the N-terminus of the C-terminal domain of the envelope protein,
optionally, the envelope protein is an envelope G glycoprotein or a mutant of envelope G glycoprotein of vesicular stomatitis virus.

21. (canceled)

22. (canceled)


SEQUENCE LISTING (10 sequences)

SEQ ID NO: 1 - protein, 495 aa, Artificial Sequence
  The mutant of the envelope G glycoprotein
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 6 aa, Artificial Sequence
  The first connecting peptide
AAATTT

SEQ ID NO: 3 - protein, 91 aa, Artificial Sequence
  The C-terminal domain of the envelope protein
FEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 4 - protein, 339 aa, Artificial Sequence
  Synthetic protein
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 5 - DNA, 1533 bp, Artificial Sequence
  Synthetic construct the first nucleic acid molecule
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgccccaaagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaacaggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaag

SEQ ID NO: 6 - DNA, 1065 bp, Artificial Sequence
  Synthetic construct the second nucleic acid molecule
atgaagtgccttttgtacttagcctttttattcattggggtgaattgcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactgggggcggaggatccggtggaggcggaagcgggggtggaggatccgaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcaactaccaccttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaag

SEQ ID NO: 7 - protein, 16 aa, Artificial Sequence
  The signal peptide
MKCLLYLAFLFIGVNC

SEQ ID NO: 8 - protein, 242 aa, Artificial Sequence
  The amino acid sequence of anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 9 - protein, 244 aa, Artificial Sequence
  The amino acid sequence of anti-Siglec15 scFv
DIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIKGGGGSGGGGSGGGGSQVQLQQPGAELVKPGASVKMSCKASGYTFTSYWITWVIQRPGQGLEWIGDIYCGSDTMHYNEKFKNKATLTVDTSSSTAYMQLSSLTSEDSAVYYCARWWDYGSSYDYFDYWGQGTTLTVSS

SEQ ID NO: 10 - protein, 341 aa, Artificial Sequence
  Synthetic protein
DIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIKGGGGSGGGGSGGGGSQVQLQQPGAELVKPGASVKMSCKASGYTFTSYWITWVIQRPGQGLEWIGDIYCGSDTMHYNEKFKNKATLTVDTSSSTAYMQLSSLTSEDSAVYYCARWWDYGSSYDYFDYWGQGTTLTVSSAAATTTFEHPHIQDAASQLPDDESLFFGDTGLSKNPIELVEGWFSSWKSSIASFFFIIGLIIGLFLVLRVGIHLCIKLKHTKKRQIYTDIEMNRLGK